(19) United States
(12) Patent Application Publication
(10) Pub. No.: US 2023/0176236 A1
(43) Pub. Date: Jun. 8, 2023

(54) X-RAY IMAGING DEVICE

(71) Applicant: Commissariat à l'Énergie Atomique et aux Énergies Alternatives, Paris (FR)

(72) Inventors: François Templier, Grenoble Cedex 9 (FR); Loick Verger, Grenoble Cedex 9 (FR); Eric Gros-Daillon, Grenoble Cedex 9 (FR); Sébastien Becker, Grenoble Cedex 9 (FR)

(73) Assignee: Commissariat à l'Énergie Atomique et aux Énergies Alternatives, Paris (FR)

(21) Appl. No.: 17/992,983

(22) Filed: Nov. 23, 2022

(30) Foreign Application Priority Data
Dec. 2, 2021 (FR) 2112877

(51) Int. Cl. G01T 1/20; H01L 25/16

(52) U.S. Cl. CPC G01T 1/20181; H01L 25/167

(57) ABSTRACT

An X-ray imaging device, including: a transfer substrate including electric connection elements; an array of pixels, each including a monolithic elementary chip bonded and electrically connected to elements of electric connection of the transfer substrate, and a photodiode formed on the transfer substrate and electrically connected to the elementary chip; and a scintillator coating the pixel array, wherein, in each pixel, the elementary chip includes an integrated circuit for reading from the pixel photodiode.

### CROSS REFERENCE TO RELATED APPLICATIONS

This application claims priority to French application number 2112877, filed Dec. 2, 2022, the contents of which is incorporated herein by reference in its entirety.

### TECHNICAL BACKGROUND

The present disclosure concerns an X-ray imaging device and a method of manufacturing such a device, particularly for radiography applications, for example, in the field of medical imaging.

### PRIOR ART

Among known X-ray imaging devices, indirect conversion devices and direct conversion devices can be distinguished.

Indirect conversion devices comprise an array of photodiodes adapted to capturing a light radiation, and a scintillator arranged above the array of photodiodes. In operation, the scintillator emits light as a result of the absorption of the X-rays. The light emitted by the scintillator is converter into electric charges by the photodiodes. Thus, the array of photodiodes acquires an image representative of the light distribution emitted by the scintillator, this light distribution being itself representative of the X-ray distribution received by the scintillator.

Direct conversion devices comprise a layer of a semiconductor conversion material adapted to directly converting the absorbed X-rays, into electric charges. The conversion layer is arranged above an array of elementary circuits adapted to reading the electric charges generated in the conversion material. In operation, the conversion layer generates electric charges as a result of the absorption of the X-rays. These charges are read by the array of readout circuits. Thus, the array of readout circuits directly acquires an image representative of the X-ray distribution received by the conversion material.

The forming of indirect conversion X-ray imaging devices is here more particularly considered.

### SUMMARY OF THE INVENTION

An embodiment provides an X-ray imaging device comprising:
a transfer substrate comprising electric connection elements;
an array of pixels, each comprising a monolithic elementary chip bonded and electrically connected to elements of electric connection of the transfer substrate, and a photodiode formed on the transfer substrate and electrically connected to the elementary chip; and
a scintillator coating each pixel,
   wherein, in each pixel, the elementary chip comprises an integrated circuit for reading from the pixel photodiode.

According to an embodiment, in each elementary chip, the integrated circuit for reading from the pixel photodiode is formed in CMOS technology.

According to an embodiment, in each pixel, the photodiode comprises an active stack based on an inorganic semiconductor material, for example, amorphous silicon of indium-gallium-zinc oxide.

According to an embodiment, in each pixel, the photodiode comprises an active organic photosensitive diode stack.

According to an embodiment, in each pixel, the photodiode comprises an upper electrode made of a transparent material.

According to an embodiment, in each pixel, the photodiode does not cover the elementary chip of the pixel.

According to an embodiment, in each pixel, the photodiode covers the elementary chip of the pixel.

According to an embodiment, in each pixel, the elementary chip of the pixel comprises an inorganic LED and an integrated circuit for controlling the LED.

Another embodiment provides an assembly comprising first and second stacked X-ray imaging devices such as defined hereabove.

According to an embodiment, the assembly comprises a filtering layer between the first and second devices.

Another embodiment provides a method of manufacturing an X-ray imaging device such as defined hereabove, wherein the elementary chips are collectively transferred and bonded to the transfer substrate, by means of a temporary support substrate.

According to an embodiment, the method comprises the forming of the photodiodes of the pixels on the transfer substrate before the step of collective transfer of the elementary chips onto the transfer substrate.

According to an embodiment, the method comprises the forming of the photodiodes of the pixels on the transfer substrate after the step of collective transfer of the elementary chips onto the transfer substrate.

### BRIEF DESCRIPTION OF THE DRAWINGS

The foregoing and other features and advantages will be discussed in detail in the following non-limiting description of specific embodiments in connection with the accompanying drawings, in which:

FIGS. 1A, 1B, 1C, 1D, 1E, 1F, 1G, 1H, and 1I are top and cross-section views illustrating steps of an embodiment of a method of manufacturing an X-ray imaging device according to an embodiment;

FIGS. 2A, 2B, 2C, 2D, and 2E are cross-section views illustrating steps of an example of a method of manufacturing elementary pixel chips of an X-ray imaging device according to an embodiment;

FIGS. 3A, 3B, 3C, 3D, 3E, and 3F are top and cross-section views illustrating steps of another example of a method of manufacturing an X-ray imaging device according to an embodiment;

FIG. 4 is a cross-section view illustrating a variant of the method of FIGS. 3A, 3B, 3C, 3D, 3E, and 3F;

FIGS. 5A and 5B are top and cross-section views illustrating steps of another example of a method of manufacturing an X-ray imaging device according to an embodiment;

FIGS. 6A and 6B are top and cross-section views illustrating steps of another example of a method of manufacturing an X-ray imaging device according to an embodiment; and

FIG. 7 is a cross-section view illustrating a variant of an X-ray imaging device according to an embodiment.

### DESCRIPTION OF THE EMBODIMENTS

Like features have been designated by like references in the various figures. In particular, the structural and/or functional features that are common among the various embodiments may have the same references and may dispose identical structural, dimensional and material properties.

For the sake of clarity, only the steps and elements that are useful for an understanding of the embodiments described herein have been illustrated and described in detail. In particular, the various possible applications of the described X-ray imaging devices have not been detailed, the described embodiments being compatible with all or most of known X-ray imaging applications, and more particularly applications capable of taking advantage of X-ray imaging devices of large dimensions, for example, devices having lateral dimensions greater than 10 cm and preferably greater than 20 cm. Further, the forming of the photosensitive diodes, of the electronic control circuits, and of the scintillator of the described devices have not been detailed, the forming of these elements being within the abilities of those skilled in the art based on the indications of the present disclosure. By X-rays, there is here meant, for example, radiations formed of photons having an energy in the range, for example, from 1,000 eV (electron-volts) to 20 MeV (mega-electron-volts).

Unless indicated otherwise, when reference is made to two elements connected together, this signifies a direct connection without any intermediate elements other than conductors, and when reference is made to two elements coupled together, this signifies that these two elements can be connected or they can be coupled via one or more other elements.

In the following description, when reference is made to terms qualifying absolute positions, such as terms “front”, “back”, “top”, “bottom”, “left”, “right”, etc., or relative positions, such as terms “above”, “under”, “upper”, “lower”, etc., or to terms qualifying directions, such as terms “horizontal”, “vertical”, etc., it is referred unless specified otherwise to the orientation of the cross-section views of the drawings.

Unless specified otherwise, the expressions “around”, “approximately”, “substantially” and “in the order of” signify within 10%, and preferably within 5%.

According to an aspect of the described embodiments, an X-ray imaging device comprising a transfer substrate, an array of photodetection pixels formed on the transfer substrate, and a scintillator coating the array of photodetection pixels is provided. Each photodetection pixel comprises a photodiode formed on the transfer substrate and electrically coupled or connected to electric connection elements (track, landings, electric connection terminals or pads) of the transfer substrate, and a monolithic elementary chip, bonded and electrically connected to elements of electric connection of the transfer substrate. In each pixel, the elementary chip is connected to the photodiode, for example by at least one element of electric connection of the transfer substrate. The elementary chip comprises at least one integrated circuit for reading from the pixel photodiode, preferably formed in CMOS technology.

Each elementary chip comprises a connection surface comprising a plurality of electric connection pads (also called terminals or landings) intended to be connected to the transfer substrate for the chip control. Each elementary chip comprises a connection surface comprising a plurality of electric connection pads (also called terminals or landings) intended to be connected to the transfer substrate for the chip control. The chips are transferred onto the transfer substrate, with their connection surfaces facing the connection surface of the transfer substrate, and bonded to the transfer substrate so as to connect the electric connection pads of each chip to the corresponding electric connection pads of the transfer substrate.

An advantage of the described embodiments is that they enable to obtain imaging devices of large dimensions, for example having lateral dimensions greater than 10 cm, preferably greater than 20 cm, at relatively low costs, while benefiting from the advantages of monolithic integrated circuits, for example, CMOS circuits, for the reading of the photodiodes. An advantage particularly lies in the low readout noise introduced by such monolithic integrated circuits with respect to circuits based on TFTs (“Thin Film Transistor”), formed by successive depositions of a plurality of thin layers directly on the transfer substrate. Another advantage is the gain in terms of reading rapidity, linked to the better mobility of the charge carriers in such monolithic integrated circuits with respect to TFT circuits. Further, such circuits optionally enable to implement additional functions of processing of the electric signals delivered by the photodiodes. Another advantage lies in the low bulk of monolithic elementary chips with respect to TFT circuits.

Examples of embodiment of such an X-ray imaging device will be described in further detail hereafter in relation with the drawings.

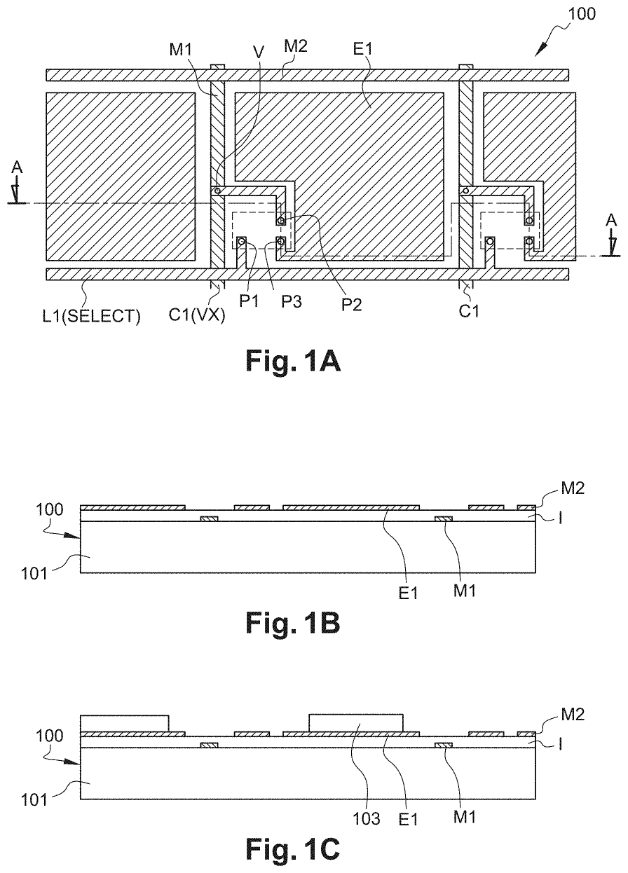

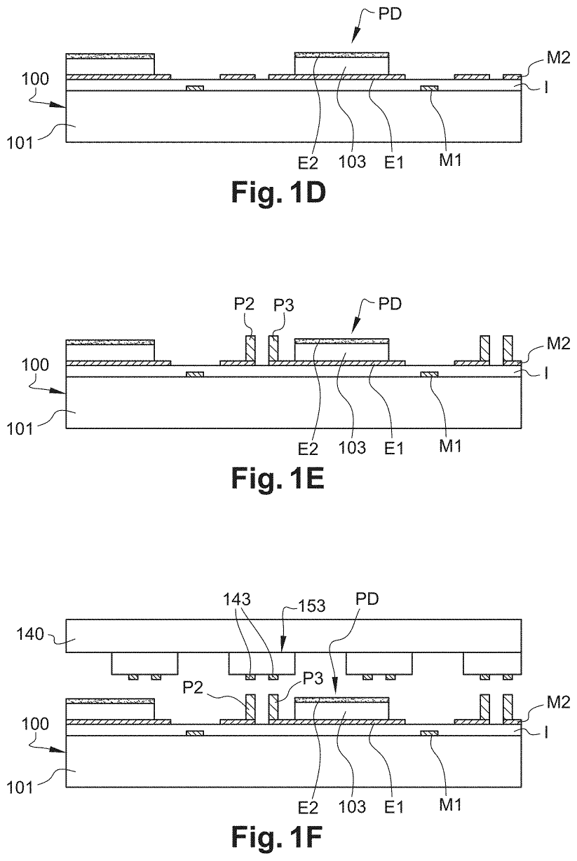

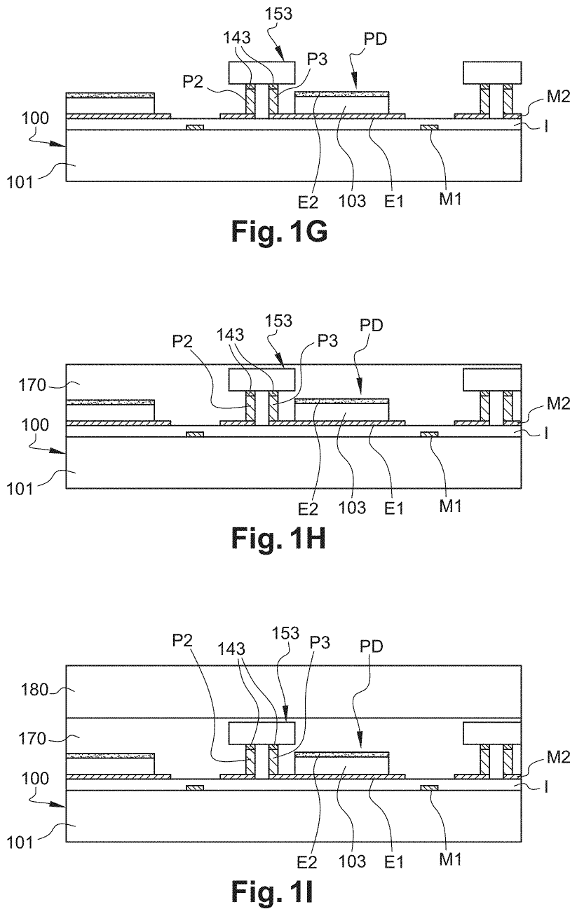

FIGS. 1A, 1B, 1C, 1D, 1E, 1F, 1G, 1H, 1I are top and cross-section views illustrating steps of an embodiment of an X-ray imaging device manufacturing method according to an embodiment.

FIG. 1A is a partial simplified top view of an example of embodiment of the transfer substrate 100 of the imaging device.

In FIG. 1A, only a portion of transfer substrate 100, corresponding to two adjacent pixels of a same row of the imaging device, has been shown.

FIGS. 1B to 1I are cross-section views of the device at different manufacturing stages, along the cross-section line A-A of FIG. 1A.

Transfer substrate 100 for example comprises a support plate or sheet 101 made of an insulating material, for example, of glass or of plastic. As a variant, support plate or sheet 101 comprises a conductive support, for example, metallic, covered with a layer of an insulating material. The transfer substrate further comprises electric connection elements, and in particular conductive tracks and conductive pads, formed on the upper surface of support plate 101. These electric connection elements are for example formed by full plate deposition and etching of a succession of conductive and insulating levels on the upper surface of support plate 101. As a variant, the electric connection elements are formed by printing (or another local deposition method) of a succession of conductive and insulating levels on the upper surface of support plate 101.

In the shown example, transfer substrate 100 comprises two conductive metal levels M1 and M2 separated by an insulating level I (not shown in FIG. 1A), and metal vias V (not shown in FIG. 1B) connecting the two metal levels M1 and M2 through insulating level I. In this example, transfer substrate 100 further comprises metal connection pads formed on upper metal level M2, intended to be connected to corresponding connection pads of the elementary chips of the pixels of the device.

Active control circuits of the display device, adapted to powering and controlling the elementary chips of the device via the electric connection elements of the transfer substrate, are for example connected to the electric connection elements of the transfer substrate at the periphery of transfer substrate 100.

As an example, the manufacturing of transfer substrate 100 comprises the three following successive deposition and etching steps.

During a first step, a conductive layer, for example metallic, for example made of titanium, of copper, or of aluminum, is deposited on the upper surface of substrate 101 and then etched to form level M1. In this example, level M1 comprises a plurality of conductive tracks substantially parallel to the column direction of the array of pixels of the imaging device (vertical direction in the orientation of FIG. 1A). More particularly, in this example, there is formed, in level M1, for each column of the imaging device, a conductive track C1 extending along substantially the entire length of the columns of the device. Each track C1 is intended to convey a signal VX representative of the quantity of charges photogenerated in the photodiodes of the pixels of the corresponding column, and thus of the light intensity received by the photodiodes of the pixels of the corresponding column.

During a second step, level M1 is covered with a layer of an insulating material, for example, silicon oxide or silicon nitride, to form insulating level I. Local openings are then etched in insulating layer I at the locations of vias V, to enable to establish electric connections between level M1 and level M2. The openings in insulating layer I are for example formed by wet etching, for example, of BHF (“Buffered Hydrofluoric Acid”) type, or by plasma etching.

During a third step, a conductive layer, for example, metallic, is deposited on the upper surface of insulating level I and then etched to form level M2. The metal layer of level M2 is preferably reflective. As an example, the metal layer of level M2 is made of aluminum. In this example, level M2 comprises a plurality of conductive tracks substantially parallel to the row direction of the array of pixels of the imaging device (horizontal direction in the orientation of FIG. 1A). More particularly, in this example, there is formed in level M2, for each row of the imaging device, a conductive track L1 extending along substantially the entire length of the rows of the device. Each track L1 is intended to convey a signal SELECT for selecting the photodiodes of the pixels of the corresponding row.

In this example, there is further formed in level M2, for each pixel of the device, a metal region E1, defining a lower electrode of the pixel photodiode.

After the third deposition step, there are formed, for each pixel, on conductive areas of metal level M2, three metal pads P1, P2, P3, intended to respectively receive three distinct connection pads of the elementary chip of the pixel. Pads P1, P2, P3 are respectively connected to the conductive track L1 of the corresponding pixel row, to the conductive track C1 of the corresponding pixel column, and to the lower electrode E1 of the photodiode of the corresponding pixel.

In practice, metal pads P1, P2, P3 may be formed immediately after the forming of level M2, or subsequently. In the example shown in the cross-section view of FIGS. 1B to 1I, metal pads P1, P2, P3 are formed after the forming of the photodiodes of the pixels (step of FIG. 1E).

FIG. 1C illustrates the structure obtained at the end of a step of forming, in each pixel, of an active photosensitive diode stack 103 on the lower electrode E1 of the pixel photodiode.

Stack 103 is for example a PIN diode stack. As an example, stack 103 is a stack based on an inorganic semiconductor material that can be deposited as a thin film on a relatively large surface area, for example, amorphous silicon, or indium gallium zinc oxide (IGZO). As a variant, stack 103 is an organic photodiode stack for example comprising an organic active semiconductor layer sandwiched between two charge transport layers (not detailed in the drawing).

As a non-limiting example, the pitch between pixels of the device in the row direction and in the column direction is in the range from 50 to 500 μm, for example from 100 to 200 μm, for example in the order of 150 μm. Preferably, the surface area (in top view) occupied by lower electrode E1 is greater than 50%, preferably greater than 70%, of the pixel surface area. Active photosensitive diode stack 103 covers substantially the entire surface area of electrode E1.

Active stack 103 is for example first continuously deposited over the entire surface of the transfer substrate, and then locally removed, for example by photolithography and etching, to only keep tiles separate from the stack, located in front of the lower electrodes E1 of the photodiodes of the pixels.

FIG. 1D illustrates the structure obtained at the end of a step of forming, in each pixel, of an upper electrode E2 on the active photosensitive diode stack 103 of the pixel. Electrode E2 is made of a transparent conductive material, for example, a transparent conductive oxide, for example, indium tin oxide (ITO). In each pixel, the stack formed by lower electrode E1, active stack 103, and upper electrode E2 defines a photodiode PD of the pixel.

As an example, the electrodes E2 of the photodiodes PD of the sensor pixels are all interconnected. In other words, the upper electrode of photodiodes PD is common to all the sensor pixels. The lower electrodes E1 of the different photodiodes PD are however distinct, to allow an individual reading of the photodiodes PD of the device.

In the shown example, active photosensitive diode stack 103 is pixelated, that is, each pixel comprises a tile formed by a portion of stack 103, laterally separated from the portions of the stack 103 of the other photosensitive diodes PD by insulating trenches. As a variant (not shown), for example in the case where active stack 103 is an active organic photosensitive diode stack, stack 103 forms a gate continuously extending over the entire pixel array, the pixelization being only performed at the level of the lower electrodes E1 of the pixels.

FIG. 1E illustrates the structure obtained at the end of a step of forming, in each pixel, of the metal connection pads P1, P2, P3 intended to be bonded and electrically connected to corresponding metal connection pads of the elementary chip of the pixel. In this example, pad P1 is formed on a conductive track portion of level M2 connected to the conductive column track C1 of the pixel, pad P2 is formed on a conductive track portion of level M2 connected, via a via V, to the row conductive track L1 of the pixel, and pad P3 is formed on a conductive track portion of level M2 connected to the lower electrode E1 of the photodiode PD of the pixel.

In the shown example, the upper surface of pads P1, P2, P3 is located at a level higher than the upper surface of the upper electrode E2 of the photodiodes PD of the pixels. In other words, the plane of the upper surface of pads P1, P2, P3 is located above the plane of the upper surface of the pixel electrodes E2.

FIGS. 1F and 1G illustrate a step of transfer, in each pixel, of an elementary control and readout chip 153 bonded and electrically connected to the metal connection pads P1, P2, P3 of the pixel.

In this example, elementary chips 153 are collectively transferred from a temporary support substrate 140 to transfer substrate 100.

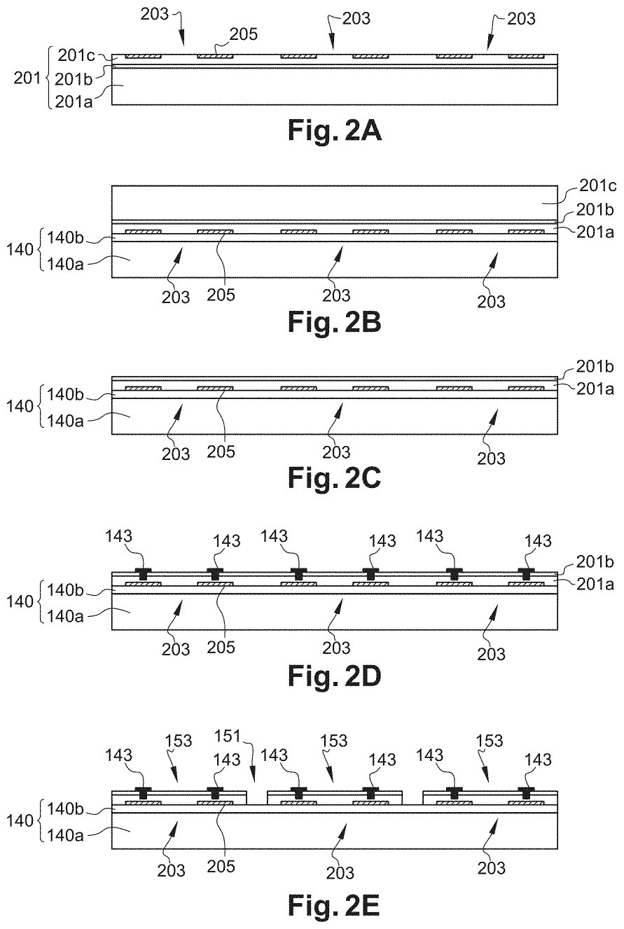

Elementary chips 153 are initially bonded to a surface of temporary support substrate 140 (lower surface in the orientation of the drawings). The structure comprising temporary support substrate 140 and elementary chips 153 is for example formed by a method of the type described hereafter in relation with FIGS. 2A to 2E.

Each elementary chip comprises at least one and preferably a plurality of MOS transistors formed inside and on top of a semiconductor substrate, for example a single-crystal silicon substrate. Elementary chips 153 are for example formed in CMOS technology. Each elementary chip is adapted to delivering, on the column conductive track C1 of the corresponding pixel (via terminal P2), a signal, for example, a voltage, representative of a light intensity received by the photodiode PD of the pixel. Chips 153 may be selected row by row, via the signal SELECT applied to the corresponding conductive track L1, to read photodiodes PD row by row during an image acquisition phase.

Elementary chips 153 are collectively transferred in front of the connection surface of transfer substrate 100, that is, its upper surface in the orientation of the drawings, by using temporary support substrate 140 as a handle (FIG. 1F).

Connection pads 143 of elementary chips 153, located on the lower surface side of said chips, are then placed into contact with the corresponding connection pads P1, P2, P3 of transfer substrate 100, and bonded to said connection pads P1, P2, P3. The bonding of the connection pads 143 of elementary chips 153 to the connection pads of the transfer substrate is for example performed by direct bonding, by thermocompression, by soldering, by means of metal microstructures (for example, micropillars) previously formed on pads 143, or by any other adapted bonding and connection method, for example by connection by means of a conductive film of AFC (“Anisotropic Conducting Film”) type.

Once bonded, by their connection pads 143, to transfer substrate 100, elementary chips 153 are separated from temporary support substrate 140, and the latter is removed (FIG. 1G), clearing the access to the illumination surface of photodiodes PD.

The pitch of elementary chips 153 on transfer substrate 100 may be greater than the pitch of elementary chips 153 on temporary support substrate 140. Preferably, the pitch of elementary chips 153 on transfer substrate 100 is a multiple of the pitch of elementary chips 153 on temporary support substrate 140. In this case, only part of chips 153 is sampled from support substrate 140 at each transfer, as illustrated in FIGS. 1F and 1G. The other chips 153 remain fastened to temporary support substrate 140 and may be used during another step of collective transfer to populate another portion of transfer substrate 100 or another transfer substrate.

FIG. 1G illustrates a step of deposition of a planarization layer 170 on the structure obtained at the end of the steps of FIGS. 1A to 1G. The material of layer 170 is a transparent dielectric material, for example, a polymer material. The material of layer 170 extends from the upper surface of the support substrate, up to a height greater than that of the upper surface of elementary chips 153. Thus, the material of layer 170 entirely covers the support substrate, photodiodes PD, and elementary chips 153. The upper surface of layer 170 is substantially planar and continuously extends over the entire surface of the pixel array.

FIG. 1I illustrates the structure obtained at the end of a step of deposition of a scintillator 180 on the upper surface of planarization layer 170. Scintillator 180 comprises a layer of a scintillation material, for example cesium iodide (CsI) in crystal form, gadolinium oxide (GadOx), or any other adapted scintillation material, that is, a material emitting light as a result of a deposition of energy by interaction with X-rays, continuously extending across the entire surface of transfer substrate 100.

It should be noted that the drawings are not shown to scale. As an example, the scintillation layer may have a thickness in the range from 200 μm to 1 mm according to the targeted applications, for example, in the order of 600 μm. The thickness of photodiodes PD is for example in the range from 1 to 10 μm, for example from 1 to 2 μm for photodiodes based on amorphous silicon, or on indium gallium zinc oxide (IGZO), and from 1 to 5 μm for organic photodiodes. The thickness of elementary chips 153 is for example in the range from 100 μm to 500 μm. The lateral dimensions of elementary chips 153 are for example in the range from 5 to 150 μm, for example from 10 to 60 μm.

Scintillator 180 is for example formed separately on a growth substrate, and then transferred onto the upper surface of passivation layer 170. As a variant, scintillator 180 is directly formed on the upper surface of passivation layer 170.

FIGS. 2A, 2B, 2C, 2D, and 2E are cross-section views illustrating successive steps of an example of a method of manufacturing the elementary chips 153 of an X-ray imaging device of the type described in relation with FIGS. 1A to 1I.

FIG. 2A schematically shows a control structure comprising a first substrate 201 inside and on top of which have been formed a plurality of elementary integrated control circuits 203, for example identical or similar, respectively corresponding to the integrated control circuits of the future elementary chips 153 of the pixels of the device.

In the shown example, substrate 201 is a substrate of SOI (“Semiconductor On Insulator”) type, comprising a semiconductor support substrate 201a, for example, made of silicon, an insulating layer 201b, for example made of silicon oxide, arranged on top of and in contact with the upper surface of support substrate 201a, and an upper semiconductor layer 201c, for example made of single-crystal silicon, arranged on top of and in contact with the upper surface of insulating layer 201b.

In this example, elementary control circuits 203 are formed inside and on top of the upper semiconductor layer 201c of substrate 201. Each elementary control circuit 203 for example comprises one or a plurality of MOS transistors (not detailed in the drawings). Elementary control circuits 203 are for example formed in CMOS technology (“Complementary Metal Oxide Semiconductor”). Each elementary control circuit 203 may comprise a circuit for reading from a photodiode of the imaging device.

FIG. 2B illustrates the structure obtained at the end of a step of transfer and of bonding of the structure of FIG. 2A onto temporary support substrate 140.

In FIG. 2B, the orientation of the structure of FIG. 2A is inverted with respect to FIG. 2A.

In this example, temporary support substrate 140 comprises a first layer 140a of a support material, for example, glass or silicon, having a thickness in the range, for example, from 200 to 700 μm, and a second thinner layer 140b made of an adhesive material of relatively low adherence to allow the selective separation of the elementary chips during the step of collective transfer of FIGS. 1F and 1G, for example a polymer material. In this example, layer 140b is arranged on top of and in contact with the upper surface of layer 140a. The structure comprising control circuits 203 is bonded to the upper surface of layer 140b by its lower surface, that is, its surface opposite to support 201a (corresponding to its upper surface in the orientation of FIG. 2A).

FIG. 2C illustrates the structure obtained after a step of removal of the support 101c of the initial SOI structure, for example by grinding and/or chemical etching, to clear the access to the upper surface of the insulating layer 201b of the SOI structure.

It should be noted that the described embodiments are not limited to the above-described example where substrate 201 is an SOI-type substrate. As a variant, substrate 201 may be solid semiconductor substrate, for example, made of silicon. In this case, at the step of FIG. 2C, substrate 201 may be thinned from its back side (upper surface in the orientation of FIG. 2C), for example by grinding. An insulating passivation layer, for example made of silicon oxide, may then be deposited on the upper surface of the thinned substrate, replacing layer 201b of the SOI substrate.

FIG. 2D illustrates the structure obtained at the end of steps of forming of contacting openings in layers 201b and 201c, and of forming of contacting metallizations 143 inside and on top of said openings. Metallizations 143 enable to take electric contacts on metal levels (not detailed in the drawings) of the interconnection stack located on the side of the lower surface of semiconductor layer 201c. Metallizations 143 are for example electrically connected to transistors of the control circuit, these transistors being themselves electrically connected or coupled to connection metallizations 205 of circuits 203.

Metallizations 143 form connection terminals of the future elementary chips of the pixels of the device, intended to be connected to corresponding connection terminals of the transfer substrate 100 of the device.

FIG. 2E illustrates the structure obtained at the end of a step of singulation of the elementary pixel chips of the device. For this purpose, trenches 151 extending vertically through layers 201b and 201c are formed from the upper surface of the structure, along sawing lines. In this example, the trenches emerge onto the upper surface of temporary support substrate 140. In top view, trenches 151 form a continuous gate laterally delimiting a plurality of elementary pixel chips 153, for example, identical or similar, each comprising an elementary control circuit 203. Trenches 151 are for example formed by plasma etching.

Elementary chips 153 are intended to be transferred onto the transfer substrate 100 of the X-ray imaging device, as has been described hereabove in relation with FIGS. 1A to 1I.

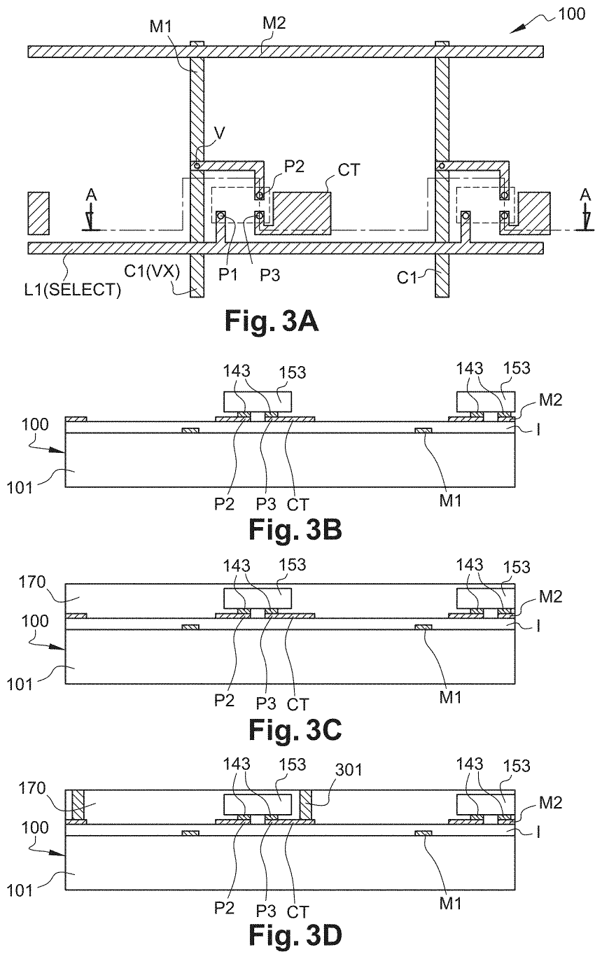

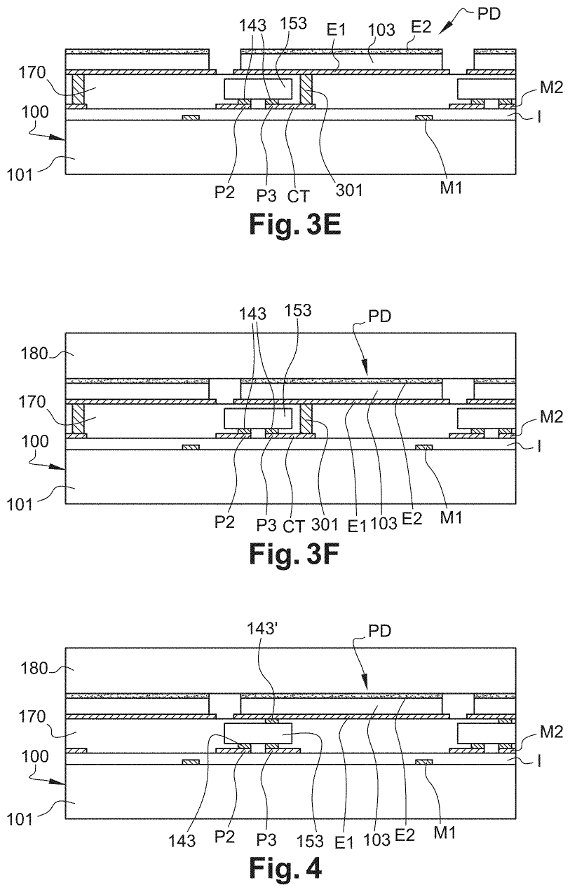

FIGS. 3A, 3B, 3C, 3D, 3E, and 3F are top and cross-section views illustrating steps of another example of a method of manufacturing an X-ray imaging device according to an embodiment.

The method of FIGS. 3A to 3F differs from the method of FIGS. 1A to 1I in that, in the method of FIGS. 3A to 3F, the elementary chips 153 of the pixels are transferred onto transfer substrate 100 before the forming of photodiodes PD, and not after as in the example of FIGS. 1A to 1I.

FIG. 3A is a partial simplified top view of an example of embodiment of the transfer substrate 100 of the imaging device.

FIGS. 3B to 3F are cross-section views along line A-A of FIG. 3A.

As in the example of FIGS. 1A to 1I, transfer substrate 100 comprises two conductive metal levels M1 and M2 separated by an insulating level I, and metal vias V connecting the two metal levels M1 and M2 through insulating level I. In this example, transfer substrate 101 further comprises metal connection areas formed on upper metal level M2, intended to be connected to corresponding connection areas of the elementary chips of the pixels of the device.

As an example, similarly to what has been described in relation with FIGS. 1A to 1I, level M1 comprises, for each pixel column of the imaging device, a conductive track C1 extending along substantially the entire length of the columns of the device, intended to convey a signal VX representative of the light intensity received by the photodiodes of the pixels of the corresponding column, and level M2 comprises, for each row of pixels of the imaging device, a conductive track L1 extending along substantially the entire length of the rows of the device, intended to convey a signal SELECT for selecting the photodiodes of the pixels of the corresponding row.

In this example, there is further formed in level M2, for each pixel, a metal region CT, defining a contacting region intended to be electrically connected to a lower electrode of the pixel photodiode. Conversely to the example of FIGS. 1A to 1I, region CT does not directly form the lower electrode of the pixel photodiode. In particular, region CT may have a surface area smaller than that of the lower electrode E1 of the pixel photodiode.

After the forming of level M2, there are formed, for each pixel, on conductive areas of metal level M2, three metal pads P1, P2, P3, intended to respectively receive three distinct connection pads of the elementary chip of the pixel. Pads P1, P2, P3 are respectively connected to the conductive track L1 of the corresponding pixel row, to the conductive track C1 of the corresponding pixel column, and to the contacting region CT on the lower electrode of the photodiode of the corresponding pixel.

As a variant, connection pads P1, P2, P3 may be directly formed by portions of level M2.

In this example, pad P1 is connected to the conductive column track C1 of the pixel via a conductive track portion of level M2, pad P2 is connected to row conductive track L1 of the pixel via a conductive track portion of level M2 and a via V, and pad P3 is connected to region CT by a conductive track portion of level M2.

FIG. 3B illustrates the structure obtained at the end of a step of transfer, in each pixel, of an elementary control and readout chip 153 bonded and electrically connected to the metal connection pads P1, P2, P3 of the pixel.

Chips 153 are for example collectively transferred from a temporary support substrate, similarly to what has been described hereabove in relation with FIGS. 1F and 1G.

FIG. 3C illustrates a step of deposition of a planarization layer 170 on the structure obtained at the end of the steps of FIGS. 3A and 3B. The material of layer 170 is a dielectric material, transparent or not, for example, a polymer material. The material of layer 170 extends from the upper surface of the support substrate, up to a height greater than that of the upper surface of elementary chips 153. Thus, the material of layer 170 entirely covers the support substrate and elementary chips 153. The upper surface of layer 170 is substantially planar and continuously extends over the entire surface of transfer substrate 100.

FIG. 3C further illustrates a step of forming, in each pixel, of a conductive via 301 extending vertically through layer 170. Via 301 is in contact, by its lower surface, with the upper surface of contact region CT. The upper surface of via 301 is flush with the upper surface of layer 170.

FIG. 3E illustrates the structure obtained at the end of the successive steps of:

forming, in each pixel, the lower electrode E1 of the pixel photodiode;
forming, in each pixel, an active photodiode stack 103 on top of and in contact with the upper surface of electrode E1; and
forming, in each pixel, the upper electrode E2 of the pixel photodiode.

Electrode E1, active stack 103, and upper electrode E2 are for example identical or similar to what has been described hereabove in relation with FIGS. 1B, 1C, and 1D.

In each pixel, lower electrode E1 is in contact, by its lower surface, with the upper surface of the pixel via 301. Thus, lower electrode E1 is electrically connected to the contacting region CT of the pixel (and thus the elementary chip 153 of the pixel) by means of via 301.

In each pixel, the stack formed by lower electrode E1, active stack 103, and upper electrode E2 defines a photodiode PD of the pixel.

As an example, the electrodes E2 of the photodiodes PD of the sensor pixels are all interconnected. In other words, the upper electrode of photodiodes PD is common to all the sensor pixels. The lower electrodes E1 of the different photodiodes PD are however distinct, to allow an individual reading of the photodiodes PD of the device.

Preferably, photodiode PD, and more particularly the active stack 103 of photodiode PD, extend above the elementary pixel chip 153.

This enables, for an equivalent pitch between pixels, to increase the photodetection surface area of the pixel with respect to the example of FIGS. 1A to 1I.

In the shown example, active photosensitive diode stack 103 is pixelated, that is, each pixel comprises a tile formed by a portion of stack 103, laterally separated from the portions of the stack 103 of the other photosensitive diodes PD by insulating trenches. As a variant (not shown), for example in the case where active stack 103 is an active organic photosensitive diode stack, stack 103 continuously extends over the entire pixel array, the pixelization being only performed at the level of the lower electrodes E1 of the pixels.

FIG. 3F illustrates the structure obtained at the end of a step of deposition of a scintillator 180, for example identical or similar to that of FIG. 1I, above photodiodes PD.

In this example, scintillator 180 is directly deposited above photodiodes PD, with no intermediate planarization layer.

As a variant, a transparent planarization layer may be deposited above photodiodes PD before the deposition of scintillator 180.

FIG. 4 is a cross-section view illustrating a variant of the method of FIGS. 3A, 3B, 3C, 3D, 3E, and 3F.

In this variant, in each pixel, the electric connection between elementary chip 153 and the lower electrode E of the pixel photodiode PD is performed via a metal connection terminal 143′ of the elementary chip, located at the upper surface of the elementary chip. Connection terminal 143′ is flush with the upper surface of planarization layer 170. Connection terminal 143′ is in contact, by its upper surface, with the lower surface of electrode E1. Thus, the conductive via 301 and the contact metal region CT of the example of FIGS. 3A to 3F may be omitted.

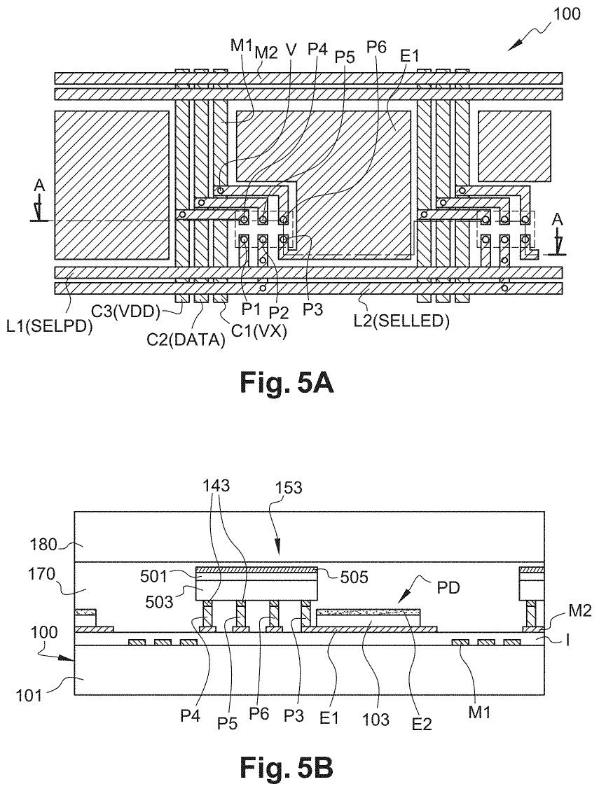

FIGS. 5A and 5B are top and cross-section views illustrating steps of another example of a method of manufacturing an X-ray imaging device according to an embodiment.

FIG. 5A is a partial simplified top view of an example of embodiment of the transfer substrate 100 of the imaging device.

FIG. 5B is a cross-section view of the device along cross-section line A-A of FIG. 5A.

The method of FIGS. 5A and 5B comprises steps identical or similar to the steps of the method of FIGS. 1A to 1I. These steps will not be detailed again hereafter and only the differences with respect to the method of FIGS. 1A to 1I will be highlighted.

The example of FIGS. 5A and 5B differs from the example of FIGS. 1A to 1I mainly in that, in the example of FIGS. 5A and 5B, in each pixel, each elementary chip 153 placed on the transfer substrate comprises not only an integrated circuit for controlling and reading from the photodiode PD of the pixel, but also an inorganic light-emitting diode (LED), and an integrated circuit for controlling the LED.

The integration of a LED in elementary chip 153 advantageously enables to implement, between two image acquisition phases, a step of resetting of photodiodes PD by application of a light flash on photodiodes PD.

Elementary chips 153 are monolithic pixel chips, for example, of the type described in the previously-filed patent applications WO2017089676, EP3401958, and WO2018185433. Each chip comprises a LED 501 and an elementary control circuit 503 placed against and electrically connected to the LED. Control circuit 503 is for example made in CMOS technology. Circuit 503 for example comprises a circuit for controlling and reading from the photodiode PD of the pixel, and a circuit for controlling the LED.

In this example, LED 501 covers the upper surface of elementary control circuit 503. Circuit 503 comprises connection terminal 143 on its lower surface side.

In the shown example, LED 501 is covered, on its upper surface side, with an opaque or reflective layer 505, for example made of metal. Layer 505 enables to direct the light emitted by the LED towards the photodiode PD of the pixel. Layer 505 for example forms the upper electrode of LED 501.

In the example of FIGS. 5A and 5B, planarization layer 170 (FIG. 5B) is made of a transparent material.

To enable to read from the photodiodes PD of the pixels and to control the LEDs 501 of the pixels in transmit mode, the transfer substrate 100 of the example of FIGS. 5A and 5B comprises a number of conductive tracks and of connection metallizations greater than that of the example of FIGS. 1A to 1I. Further, elementary chips 153 comprise a number of connection terminals greater than what has been previously described.

In the shown example, each elementary chip comprises six connection terminals intended to be respectively connected to six metal connection pads P1, P2, P3, P4, P5, P6 of transfer substrate 100.

As an example, level M1 comprises, for each pixel column of the imaging device, three conductive column tracks C1, C2, C3 intended to respectively convey a signal VX representative of the light intensity received by the photodiodes of the pixels of the corresponding column, a signal DATA for controlling the LEDs of the pixels of the corresponding column, and a signal VDD for powering the LEDs of the pixels of the corresponding column. In this example, level M2 comprises, for each pixel column of the imaging device, two row conductive tracks L1 and L2 intended to respectively convey a signal SELPD of selection of the photodiodes of the pixels of the corresponding row, and a signal SELLED for selecting the LEDs of the pixels of the corresponding row.

Metal pads P1, P2, P3, P4, P5, P6 are formed on conductive areas of metal level M2 and are intended to respectively receive six distinct connection pads of the elementary pixel chip. Pads P1, P2, P3, P4, P5, P6 are respectively connected to the conductive track L1 of the corresponding pixel row, to the conductive track L2 of the corresponding pixel row, to the electrode E1 of the photodiode PD of the corresponding pixel, to the conductive track C3 of the corresponding pixel column, to the conductive track C4 of the corresponding pixel column, and to the conductive track C5 of the corresponding pixel column.

In the example of FIGS. 5A and 5B, similarly to what has been described hereabove in relation with FIGS. 1A to 1I, elementary chips 153 are bonded and electrically connected to transfer substrate 100 before the forming of the photodiodes PD of the pixels.

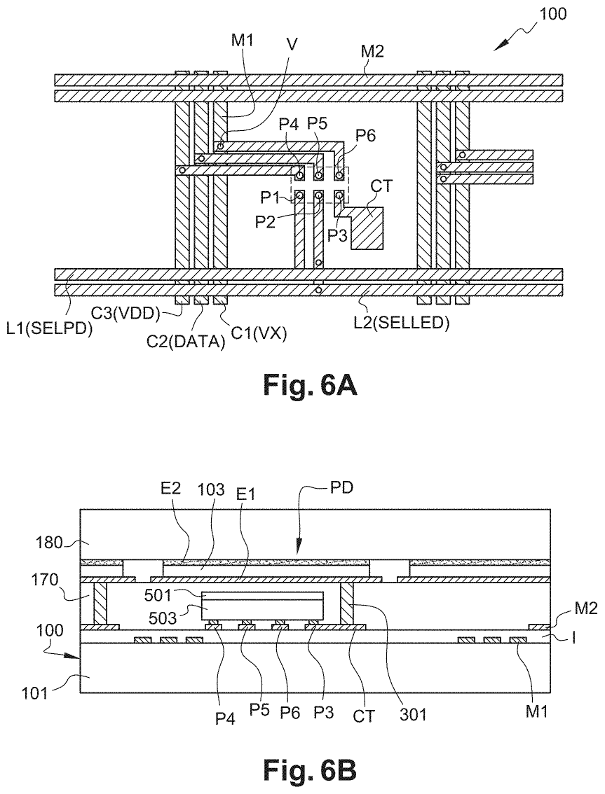

FIGS. 6A and 6B are top and cross-section views illustrating steps of an alternative embodiment of the method of FIGS. 5A and 5B, where the elementary chips are bonded and electrically connected to the transfer substrate after the forming of photodiodes PD, similarly to what has been described hereabove in relation with FIGS. 3A to 3F.

It should be noted that in this variant, the reflective layer 505 of FIG. 5B may be omitted or replaced with a transparent layer, for example, a transparent conductive layer, for example made of ITO, forming the upper electrode of LED 501. Indeed, in each pixel, the elementary LED 501 of the pixel is directly located under the photodiode PD of the pixel.

In the example of FIGS. 6A and 6B, planarization layer 170 (FIG. 6B) is made of a transparent material.

It should be noted that the variant of FIGS. 6A and 6B may be combined with the variant of FIG. 4, in which case the connection elements CT and 301 of FIG. 6B may be omitted.

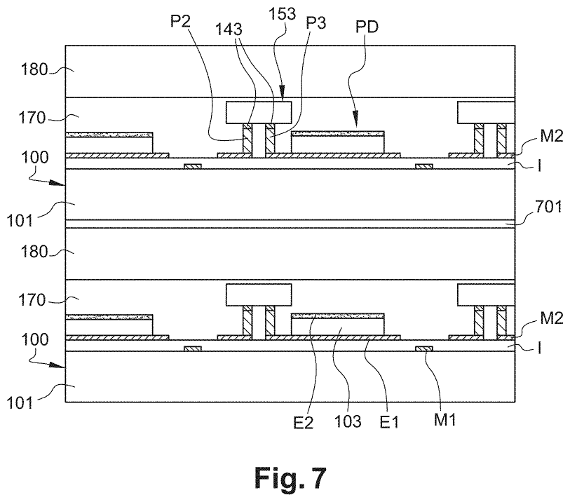

FIG. 7 is a cross-section view illustrating another alternative embodiment of an X-ray imaging device according to an embodiment. In this example, the device comprises two stacked devices of the type described in relation with FIG. 1I.

The device of FIG. 7 enables to perform dual-energy X-ray imaging, also called color X-ray imaging, that is, to respectively image a first energy level, called low-energy level (BE), by means of the upper imaging device, and a second energy level, called high-energy level (HE), by means of the lower imaging device. As an example, the upper imaging device is adapted to detecting radiations having an energy level in the range from 1 keV to 140 keV, for example, from 40 keV to 80 keV, for example in the order of 60 keV in average, and the lower imaging device is adapted to detecting radiations having an energy level in the range from 60 keV to 140 keV, for example from 80 keV to 120 keV, for example in the order of 100 keV in average.

In the shown example, an interface layer 701 is arranged between the lower surface of the support substrate 101 of the upper device and the upper surface of the scintillator 180 of the lower device. The thickness of the support substrate of the upper device is preferably relatively small to limit the absorption of high-energy photons. As an example, the thickness of the support substrate of the upper device is smaller than the thickness of the support substrate of the lower device.

Interface layer 710 may comprise a filtering layer adapted to filtering the low-energy radiation so that only the high-energy radiation reaches the lower imaging device. The filtering layer is for example a metal layer, for example, continuous, for example, made of copper or of aluminum, for example having a thickness in the range from 0.1 to 0.4 mm. The filtering layer enables to improve the spectral separation between the two imaging devices.

As a variant, interface layer 701 may be omitted.

The embodiment of FIG. 7 may of course be combined with all the previously-described variants.

Various embodiments and variants have been described. Those skilled in the art will understand that certain features of these various embodiments and variants may be combined, and other variants will occur to those skilled in the art. In particular, the described embodiments are not limited to the examples of dimensions and of materials mentioned in the disclosure.

Further, examples of embodiment where a scintillator 180 covers an array of pixels, each comprising a monolithic elementary chip and a photodiode, have been described hereabove. As a variant, in each pixel, the detector comprises an active detection stack based on a scintillator material 180 adapted to directly converting X photons into light photons, for example, a material from the group comprising cesium iodide (CsI:T1) or GADOX (GD2O2S:Tb), which light photons then interact with the pixel photodiode to generate electrons.

What is claimed is:

1. X-ray imaging device, comprising:
a transfer substrate comprising electric connection elements;
an array of pixels, each comprising a monolithic elementary chip bonded and electrically connected to elements of electric connection of the transfer substrate, and a photodiode formed on the transfer substrate and electrically connected to the elementary chip; and
a scintillator coating each pixel,
wherein, in each pixel, the elementary chip comprises an integrated circuit for reading from the pixel photodiode.

2. Device according to claim 1, wherein, in each elementary chip, the integrated circuit for reading from the pixel photodiode is formed in CMOS technology.

3. Device according to claim 1, wherein, in each pixel, the photodiode comprises an active stack based on an inorganic semiconductor material, for example, amorphous silicon or indium-gallium-zinc oxide.

4. Device according to claim 1, wherein, in each pixel, the photodiode comprises an active organic photosensitive diode stack.

5. Device according to claim 1, wherein, in each pixel, the photodiode comprises an upper electrode made of a transparent material.

6. Device according to claim 1, wherein, in each pixel, the photodiode does not cover the elementary chip of the pixel.

7. Device according to claim 1, wherein, in each pixel, the photodiode covers the elementary chip of the pixel.

8. Device according to claim 1, wherein, in each pixel, the elementary chip of the pixel comprises an inorganic LED and an integrated circuit for controlling the LED.

9. Assembly comprising first and second stacked X-ray imaging devices according to claim 1.

10. Assembly according to claim 9, comprising a filtering layer between the first and second devices.

11. Method of manufacturing an X-ray imaging device according to claim 1, wherein the elementary chips are collectively transferred and bonded to the transfer substrate, by means of a temporary support substrate.

12. Method according to claim 9, comprising the forming of the photodiodes of the pixels on the transfer substrate before the steps of collective transfer of the elementary chips onto the transfer substrate.

13. Method according to claim 9, comprising the forming of the photodiodes of the pixels on the transfer substrate after the step of collective transfer of the elementary chips onto the transfer substrate.
